(19) United States
(12) Patent Application Publication
(10) Pub. No.: US 2009/0292200 A1
(43) Pub. Date: Nov. 26, 2009

(54) APPARATUS AND METHOD FOR MARKING AN IRRADIATION FIELD ON THE SURFACE OF A PATIENT'S BODY

(75) Inventors: Johann Kindlein, Lueneburg (DE); Tim Thurn, Lueneburg (DE)

Correspondence Address: VIDAS, ARRETT & STEINKRAUS, P.A., SUITE 400, 6640 SHADY OAK ROAD, EDEN PRAIRIE, MN, 55344, US

(73) Assignee: LAP GmbH Laser Applikationen, Lueneburg (DE)

(21) Appl. No.: 12/193,472

(22) Filed: Aug. 18, 2008

(30) Foreign Application Priority Data
May 24, 2008 (DE) 10 2008 025 014.7

(51) Int. Cl. A61B 5/05

(52) U.S. Cl. 600/425

(57) ABSTRACT

The present invention is related to an apparatus for marking an irradiation field on the surface of the patient's body, which was produced by means of a virtual 3D model of a patient's body, with a laser system for determining the coordinates of at least two reference points on the surface of the patient's body in a local coordinate system assigned to the apparatus, with an analysing and control unit which is realised to determine a transformation matrix for the transformation of arbitrary coordinates from the virtual coordinate system into the local coordinate system from the coordinates of the reference points in the local coordinate system and from coordinates of the reference points in a virtual coordinate system of the virtual 3D model of the patient's body, and wherein the analysing and control unit is further realised to transform coordinates from the virtual coordinate system into the local coordinate system with the transformation matrix, and to provide the transformed coordinates to the laser system. In addition, the present invention is also related to a corresponding method.

### CROSS-REFERENCE TO RELATED APPLICATIONS

Not applicable.

### STATEMENT REGARDING FEDERALLY SPONSORED RESEARCH

Not applicable.

### BACKGROUND OF THE INVENTION

The present invention is related to an apparatus and a method for marking an irradiation field, which was produced by means of a virtual 3D model of a patient's body, on the surface of the patient's body. Such irradiation fields represent desired intersection fields of a therapeutic beam, mostly of ionizing radiation and used for cancer treatment in particular, with the surface of a patient's body. In this, usually plural rays are directed to the body to be treated from different directions, so that they intersect in an isocentre. Here acts the summed-up radiation dose, and the adverse effect to the surrounding tissue is minimised. As the first step of a radiation therapy, a tomography mapping, computer tomography (CT) for instance, is established of a body region of interest of the patient which is positioned and fixed on a positioning aid, like a treatment table, using special positioning and fixation aids. In this, a reference point for the tomography mapping is fixed by marking the skin surface of the patient with the laser system in the reference position, mostly the zero position. Based on the tomography mapping, a 3D-model of the body region of interest of the patient's body is made and the tumour to be treated is localised. In the context of the irradiation planning, the target volumes are contoured, the dose of the irradiation is calculated and in particular, the number and location of the radiation fields are determined, so that the tumour is irradiated as desired.

The subsequent simulation is the transfer of the coordinates (position and form) of the calculated irradiation fields from the calculated model to the real patient, and usually it comprises a marking of the irradiation fields on the skin of the patient, with a pencil for instance. Before the actual irradiation, the correct position of the irradiation fields can be examined by means of a light field of the irradiation apparatus representing the treatment beam. The marking process is the transfer of coordinates from the static 3D patient model to the living patient. Because in this it is dealt with living human beings with soft body structures and not with rigid bodies, an accurate and reproducible positioning is complicated with very strongly ill and adipose patients in particular.

Laser marking systems are used for marking. A known laser marking system is described in DE 44 21 315 A1 or DE 195 24 951 A1, the entire contents of which is incorporated herein by reference, and it consists of five motor movable lasers altogether, which are mounted in the room of the tomography apparatus. Two lasers adjustable in the height, which project a horizontal line along the treatment table accommodating the patient during the tomography, are situated at the right and at the left side of the treatment table, respectively. The remaining three lasers are mounted in a plate or holder, which is situated above the table. One laser is movable transversely to the table's long side direction and projects a line along the table. Two of the lasers in the plate or holder are coupled with each other and project a common line transversely to the table's longitudinal axis. By doing so, two lasers are coupled for the purpose that by the lateral arrangement along the patient axis, even such coordinates can be represented on the skin which otherwise would be shaded below the transverse diameter of the patient by the patient. Such a laser system is commercialised by the applicant under the name “Dorado CT4”.

For marking the calculated irradiation fields with the known laser marking system, there is at first a positioning and fixation of the patient in a position which coincides with the position of the patient in the production of the tomography scan as far as possible. In order to examine the positioning of the patient, after producing the reference plane (laser is in the reference position, mostly the zero point position), another tomography (CT for instance) is performed. In the case that greater deviations are found in this position examination, the positioning and the fixation of the patient have to be repeated or corrected, respectively. For this purpose, the treatment table accommodating the patient can be positioned correspondingly anew. After completion of the position examination, the markings on the patient's skin can be performed with the aid of the laser marking system. For this purpose, the coordinates of the desired irradiation fields calculated before are provided to the laser marking system. The coordinates can then be approached successively by means of the laser marking system, and thus they may be indicated for an operator and then they can be manually marked on the skin, by means of a pencil for instance.

In the procedure known from the state of the art, it is disadvantageous that the marking due to the position examination has to take place by means of another tomography scan in the room of the tomography apparatus. This occupies valuable time in this room. In addition, a radiation load for the patient is accompanied with the second position examination by means of a tomography scan.

Proceeding from the clarified state of the art, the present invention is based on the objective to provide an apparatus and a method of the kind indicated in the beginning, by which the marking is possible in a simple and cost saving way, wherein the danger for the health of the patient is minimised.

### BRIEF SUMMARY OF THE INVENTION

On the one hand, the present invention resolves the objective by an apparatus for marking an irradiation field on the surface of a patient's body, which was produced by means of a virtual 3D model of the patient's body, with a laser system for determining the coordinates of at least two reference points on the surface of the patient's body in a local coordinate system assigned to the apparatus with an analysing and control unit, which is realised to determine a transformation matrix for the transformation of arbitrary coordinates from the virtual coordinate system into the local coordinate system from the coordinates of the reference points in the local coordinate system and from coordinates of the reference points in a virtual coordinate system of the virtual 3D model of the patient's body, and wherein the analysing and control unit is further realised to transform coordinates from the virtual coordinate system into the local coordinate system with the transformation matrix, and to provide the transformed coordinates to the laser system.

On the other hand, the present invention resolves the objective by a method for marking an irradiation field on the surface of the patient's body, which was produced by means of a virtual 3D model of a patient's body, wherein the coordinates of at least two reference points on the surface of the patient's body are determined in a local coordinate system assigned to the apparatus, wherein a transformation matrix for the transformation of arbitrary coordinates from the virtual coordinate system into the local coordinate system is determined from the coordinates of the reference points in the local coordinate system and from coordinates of the reference points in a virtual coordinate system of the virtual 3D model of the patient's body, and wherein coordinates from the virtual coordinate system are transformed into the local coordinate system with the transformation matrix, and are provided for the marking. The marking system may be a laser marking system in particular.

The analysing and control unit can be realised to transform the coordinates of at least the irradiation isocentre from the virtual coordinate system into the local coordinate system, and to provide the transformed coordinates to the laser system. The isocentre of a tumour to be irradiated in the context of a cancer treatment is designated as the irradiation isocentre in this. During the irradiation, an irradiation apparatus used for the irradiation (a linear accelerator for instance) rotates around this isocentre. This isocentre, situated in the interior of the patient, is usually marked by three points on the skin of the patient. These points serve then for positioning the patient on the irradiation apparatus. When the coordinates of these points are determined as the reference points for instance, the transformation matrix can be determined on this basis. The analysing and control unit can be further realised to transform further coordinates from the virtual coordinate system into the local coordinate system with the transformation matrix, and to provide the transformed coordinates to the laser system. For instance, the coordinates of the outline and/or the surface and/or the form of the irradiation field can be transformed from the virtual coordinate system into the local coordinate system, and be provided for marking. The irradiation field can be a desired intersection field of a therapy beam used for cancer treatment (ionising radiation for instance) with the skin of the patient, calculated for a virtual 3D-model of a patient and produced on the basis of a tomography mapping (CT-mapping for instance). Of course, plural irradiation fields can be envisioned for marking in this. The 3D-model may represent a region of interest of the patient's body or even the complete body. The coordinates are three-dimensional coordinates in particular.

In contrast to the state of the art, the basic idea of the present invention is not to position the patient in the marking room with the laser system via displacement of the treatment table and of the patient such that he/she takes the same position again as in the tomography mapping for the irradiation planning. Instead, according to the present invention, the coordinates of the irradiation fields to be marked are matched to the (new) position of the patient in the marking room. The patient himself/herself can be positioned arbitrarily in wide limits and has not to be matched in an awesome way in his/her positioning due to the coordinate transformation. The patient can be positioned with a positioning aid in the marking room. For the mentioned reasons, the same has not necessarily to be movable.

The coordinates of the reference points approached in the marking room are known in the local coordinate system of the apparatus after being approached with the laser lines. In addition, the coordinates of the reference points are also known in the virtual coordinate system of the virtual 3D-model of the patient's body, which was produced in the context of the irradiation planning. On this basis, the transformation matrix can be determined. In this, the transformation matrix is determined by means of a comparison of the reference points, and of the transformation of the reference points from the virtual coordinate system into the local coordinate system in particular. By means of the reference points, the patient serves as a transition between the 3D-model of his/her body produced in the context of the irradiation planning and his/her body in the position taken in the marking room. The transformation matrix maps the 3D-model of the patient, and the surface of the 3D-model in particular as well as the corresponding irradiation fields, from the virtual coordinate system into the local coordinate system. In this, a new positioning of the patient in the marking room with respect to his/her position which was taken in the production of the virtual 3D-model of his/her body is taken into account via the reference points. So to speak, the reference points serve as a “bridge” between the position of the patient taken in the production of the virtual 3D-model and the position of the patient in the marking.

According to the present invention, marking the patient is possible in another room than that one of the tomography apparatus (CT apparatus, for instance) used for the production of the virtual 3D-model. Through this, no valuable time in the tomography room is required for marking. The marking room without tomography apparatus proposed in this manner is relatively inexpensive, because it has not to fulfil the severe requirements of radiation protection. In addition, the patient is not exposed to further radiation load in the context of the marking of the present invention, because new tomography mapping is not necessary.

Of course, the laser system can also be realised for the determination of more than two reference points on the surface of the patient's body in the local coordinate system, three reference points for instance. The number of the reference points to be determined for the determination of the transformation matrix depends on the number of degrees of freedom of the patients movement between his/her position in the production of the virtual 3D-model and his/her position in the marking. In principle, a three-dimensional translation and a three-dimensional rotation of the patient are possible. For determining the matrix, a so-called “matching algorithm” is used. From the position change of the patient in the respective degrees of freedom, the necessary transformation from the position of the patient in the production of the virtual 3D-model into the position of the patient in the marking is determined. From this result the translation parameters t_{X}, t_{Y}, t_{Z} in the three space directions as well as the rotational angles α, β, γ for the three possible rotations around the corresponding X-, Y- and Z-axis of the three space directions. The transformation matrix for the correction of the irradiation field coordinates is as follows in its general form:

$$K = ( \begin{matrix}{r}_{11} & {r}_{12} & {r}_{13} & {t}_{X} \\ {r}_{21} & {r}_{22} & {r}_{23} & {t}_{Y} \\ {r}_{31} & {r}_{32} & {r}_{33} & {t}_{Z} \\ 0 & 0 & 0 & 1\end{matrix} )$$

In this, the parameters t_{X}, t_{Y}, t_{Z} represent the three-dimensional translation of the patient's body between the virtual and the local coordinate system. The other parameters r₁₁ to r₃₃ represent the three-dimensional rotation of the patient's body. This correction matrix can then be applied to each coordinate point of the irradiation field coordinates which were calculated in the context of the virtual irradiation planning. Seen from the mathematical point, we have a 3D-transformation of a not deformable body in a homogeneous transformation matrix, consisting of rotation and translation.

The general form of the transformation looks like the following:

$$( \begin{matrix}{X}^{′} \\ {Y}^{′} \\ {Z}^{′} \\ 1\end{matrix} ) = ( \begin{matrix}{r}_{11} & {r}_{12} & {r}_{13} & {t}_{X} \\ {r}_{21} & {r}_{22} & {r}_{23} & {t}_{Y} \\ {r}_{31} & {r}_{32} & {r}_{33} & {t}_{Z} \\ 0 & 0 & 0 & 1\end{matrix} ) \cdot ( \begin{matrix}X \\ Y \\ Z \\ 1\end{matrix} )$$

$$or$$

$${P}^{′} = R \cdot P + T$$

In this, the transformed three-dimensional coordinates of for instance the irradiation field are designated by X′, Y′ and Z′ or P′, respectively. X, Y and Z or P, respectively, designate the coordinates of the irradiation field in the virtual coordinate system. R represents the rotation. When the transformation parameters are not known, an equation system with 6 linearly independent unknown variables altogether results from the matrix product for each three-dimensional marking point on the surface of the patient's body, namely three rotational angles α, β, γ and the translational parameters t_{X}, t_{Y}, t_{Z}. Thus, the determination of three three-dimensional reference points on the surface of the patient's body in the local coordinate system is necessary for the determination of the transformation matrix in this case. In the case of a rotation of the body around only one angle y and a translation in only one horizontal plane along the axis X and Y, the transformation is simplified into the following from:

$$( \begin{matrix}{X}^{′} \\ {Y}^{′} \\ 1\end{matrix} ) = ( \begin{matrix}{r}_{11} & {r}_{12} & {t}_{X} \\ {r}_{21} & {r}_{22} & {t}_{Y} \\ 0 & 0 & 1\end{matrix} ) \cdot ( \begin{matrix}X \\ Y \\ 1\end{matrix} )$$

$$\underline{or}$$

$$\underline{{P}^{′} = R \cdot P + T}$$

When the translational parameters are known, only two reference points have then to be approached for the determination of the matrix. As the method for determining the transformation matrix is iterative, an overdetermination of the equation system due to the determination of more reference points than necessary can lead to an increased accuracy and it may be advantageous for this reason.

The transformed coordinates can be provided to the laser system subsequently. Then, the markings of the irradiation fields and of the intersection points with the skin of the central beam, which runs through the isocentre, can take place on the skin of the patient, for instance manually by means of a pencil. Then, the form and position of the irradiation field marked in this way corresponds to the form and position of the fields calculated in the context of the irradiation planning for the new position of the patient. The analysing and control unit can be realised to drive the laser system such that the transformed coordinate points are approached one after the other with at least one laser beam. The approach to the coordinate points can take place via a remote control, for instance. Besides to the coordinates of the outline of the irradiation field, in particular the coordinates of the intersection points with the skin of the central beam, which runs through the isocentre, can be transformed and be provided to the marking device for the marking.

The marking device has at least one laser and can be realised to be combined with the analysing and control unit. The analysing and control unit has a suitable software for performing its tasks. In order to be able to approach the reference points in a simple way, they can be marked on the patient when the 3D-model is made, and for instance, they can indicate the isocentre of a tomography apparatus (CT) which was used for making the virtual 3D-model. The marking region of the patient can be situated in the zero point of the local coordinate system of the apparatus.

The reference points possibly applied to the skin of the patient as main marking points in the context of a first marking in a tomography room can represent the intersection points of the axis of the coordinate system assigned to the patient and the tomography system with the skin surface of the patient. By doing so, in this first marking of the reference points on the patient, the zero point of the coordinate system assigned to the patient can be coincident with the zero point of the coordinate system of the tomograph. As explained above, admittedly the zero point of the patient coordinate system in the marking position may be different from the zero point of the marking system, that is to say of the local coordinate system of the apparatus. This difference is determined by the determination of the reference points and is taken into account in the transformation matrix.

The laser system may comprise at least two lasers, each one thereof generating one laser line at a time. The coordinates of the reference points are known in the virtual coordinate system. The determination of the local coordinates of the reference points can be performed in a particularly simple manner through a determination of the translation of the laser lines from the zero point of the local coordinate system to the reference points. The zero point of the local coordinate system is known. The coordinates of the approached reference points are then also known by means of the translation. A corresponding position feedback takes place by the laser system. In order to make the reference points visible for instance in the virtual 3D-model of the patient's body produced in the context of a CT mapping too, contrast marks (heavy metal marks) can be applied to the patient at the reference points.

After the tomography mapping and the first marking of the patient at the reference points, a coordinate system was assigned to the patient herself/himself too.

In an arbitrary positioning of the patient, this patient coordinate system does not have to coincide with the isocentre of the tomography apparatus. The patient can be subjected to a translation as well as to a rotation with respect to the initial position. However, inasmuch as the coordinates of a sufficient number of reference points in the local coordinate system and the virtual coordinates of the reference points in the virtual coordinate system are known, the transformation matrix can be determined unambiguously.

According to a further embodiment, the laser system can comprise at least two laser projection devices, wherein the reference points can be determined on the patient's surface by an intersection of two laser beams at a time which are generated by the laser projection devices. It is then possible to realise the analysing and control unit for the purpose to drive the laser projection devices such that the transformed coordinates can be projected to the three-dimensional surface of the patient's body while at least one laser beam generated by at least one of the laser projection devices can be guided along the transformed coordinate points on the surface of the patient's body sufficiently rapidly, so that the impression of a closed contour (around the irradiation field, for instance) results on the surface. The representation of irradiation fields is the subject matter of the parallel German Patent Application of the assignee with the file number 10 2008 012 496.6. The position of the projection devices in the room is known and defined. For this purpose, the reference points can be marked by means of reflectors on the patient, for instance. When two projector beams are now directed to one reflector point, the position (3D-coordinates) of the point in 3D-space can be determined by means of the intersection point of the beams. From the determination of the local coordinates of all reference points which are also known in the virtual coordinate system of the virtual 3D-model of the patient's body, the elements of the transformation matrix can be determined in turn. The projection devices can have at least two rotatable (galvanometric) mirrors, by which the laser beams are reflected to the surface of the patient's body and can be guided to the reference points or along the outline of the irradiation field, respectively. By means of the projection of the contour to the surface, the irradiation field as well as the intersection points with the skin of the central beam, which runs through the isocentre, can in turn be marked on the skin by hand with a pencil, for instance.

A surface mapping system can be provided, by which a local 3D-model of the patient's body can be produced in the local coordinate system. Such a system may be a laser or a light strip system, for instance. According to a further embodiment of the present invention, the laser system has at least one laser generating a laser line and at least one detector device, wherein the analysing and control unit is realised to guide a laser line successively across the surface of the patient's body, to detect the laser light reflected by the patient's body by means of the detector device and to produce a local 3D-model of the patient's body in the local coordinate system from the detected laser light, with the triangulation method for instance. Of course, even a 3D-model of a part of interest of the patient's body can be produced in this. In particular, two lasers each one thereof generating one laser line at a time can be provided, which are coupled with each other in order to be able to reach every region of the patient's body. As the detector device, it may be dealt with a camera in a particularly simple manner. Laser or light intersection methods based on triangulation are per se known. In these, a camera and a fixed laser plane is mostly used. At the place where the light hits the object to be mapped, thus in the visible intersection plane between laser plane and object surface, the light beams are diffusely reflected back into the room. The reflected light is acquired by a laterally offset camera. Through a calibration of the camera and an additional calibration, in which the arrangement of camera and laser plane with respect to each other is determined, a calculation of the coordinates of the surface points illuminated by the laser light can be performed by a suitable analysing software. From this, a two-dimensional information about the shape of the object is obtained. When a three-dimensional mapping of the object is desired, either the laser line has to be moved across the object's surface in a defined manner, or the object through the measurement region. Irrespective of the method used, the arrangement of laser plane and camera has to be known for every point in time of the measurement.

The laser plane can be moved by motor along the longitudinal axis of a positioning table accommodating the patient, for instance. The laser scan may be performed by guiding a laser line which is oriented transversely to the patient, for instance, across the patient or the region of interest of the patient, respectively. By concomitant acquisition of the reflected laser radiation with the detector device, we have a laser scanner. Thus, in this embodiment of the present invention, a plurality, an infinite number in particular, of reference points is detected by producing a local 3D-model of the patient's body. In this, all the acquired surface points of the body may be reference points. These surface points are in turn also known as reference points in the virtual coordinate system of the virtual 3D-model. The 3D-models of the patient in the local coordinate system produced in doing so may also be provided as reference surfaces for a positioning check of the patient to other systems, like diagnostic or treatment apparatuses for instance.

The transformation matrix can then be determined by the analysing and control unit from a comparison of the local 3D-model of the patient's body with the virtual 3D-model of the patient's body. Thus, the local 3D-model produced by the laser scan is compared with the virtual 3D-model from the irradiation planning, which was produced in the context of a tomography mapping for instance. In this, the distance between the two 3D-models can be point wise measured for a plurality of points, and thus the 3D-models can be set into overlap such that a minimal distance between the two models is obtained across the overall surface (preferably distance zero). The transformation matrix is produced from the rotation/translation of the virtual 3D-model necessary for this. In this embodiment of the present invention, which is characterised by a particularly high accuracy, marking of the reference points on the skin of the patient in the context of the tomography scan is not necessarily required.

The laser system can comprise at least five lasers, each one generating one laser line at a time, wherein two lasers adjustable in the height are provided on the sides of a positioning table for the patient, which each one at a time project one horizontal line along the positioning table, and three lasers are provided above the positioning table, wherein one laser is arranged to be movable transversely to the longitudinal direction of the positioning table and projects a laser line in the longitudinal direction of the positioning table, and wherein the two other lasers are arranged to be movable in the longitudinal direction of the positioning table and project a common laser line transversely to the longitudinal direction of the positioning table, being coupled with each other. According to the present invention, this per se known (DE 44 21 315 A1 or DE 195 24 951 A1, the entire contents of which is incorporated herein by reference) system, marketed by the assignee under the name “Dorado CT 4” can be used in a new manner. For instance, the two coupled lasers arranged above the table can be used for the laser scanner. By the coupling of the two lasers and the lateral arrangement along the patient's axis on the treatment table, even such coordinates can be represented on the skin which otherwise would be shaded by the patient himself/herself below the transverse diameter of the patient. Then, the system has only to be completed by a corresponding detector device, a camera for instance.

The apparatus of the present invention can be arranged in another room than a tomography apparatus used for determining the virtual 3D-model of the patient's body. Through this, valuable time in the tomography room is saved. The same positioning and fixation aids for the patient can be provided in both rooms in this case. Of course, the apparatus of the present invention may also be arranged in the same room as a tomography device which is used to produce the virtual 3D-model (a CT device, for instance).

When the reference points are marked on the skin of the patient's body in the context of the tomography scan and of the production of the virtual 3D-model in particular, fixed room lasers can be used for the first marking in the tomography room, which are installed such that they correspond to the isocentre of the tomograph. After the indication by the laser, marking of the reference points on the skin of the patient can be performed with a colour pencil and at the same time with contrast parts (balls or crosses or the like), which are visible in a tomography mapping.

The apparatus of the present invention can be suited for the execution of the method of the present invention.

### BRIEF DESCRIPTION OF THE SEVERAL VIEWS OF THE DRAWINGS

An example of the execution of the invention is explained in more detail in the following by means of figures. Schematically shown is in:

FIG. 1 an apparatus of the present invention according to a first embodiment,

FIG. 2 an apparatus of the present invention according to a second embodiment, and

FIG. 3 an apparatus of the present invention according to a third embodiment.

### DETAILED DESCRIPTION OF THE INVENTION

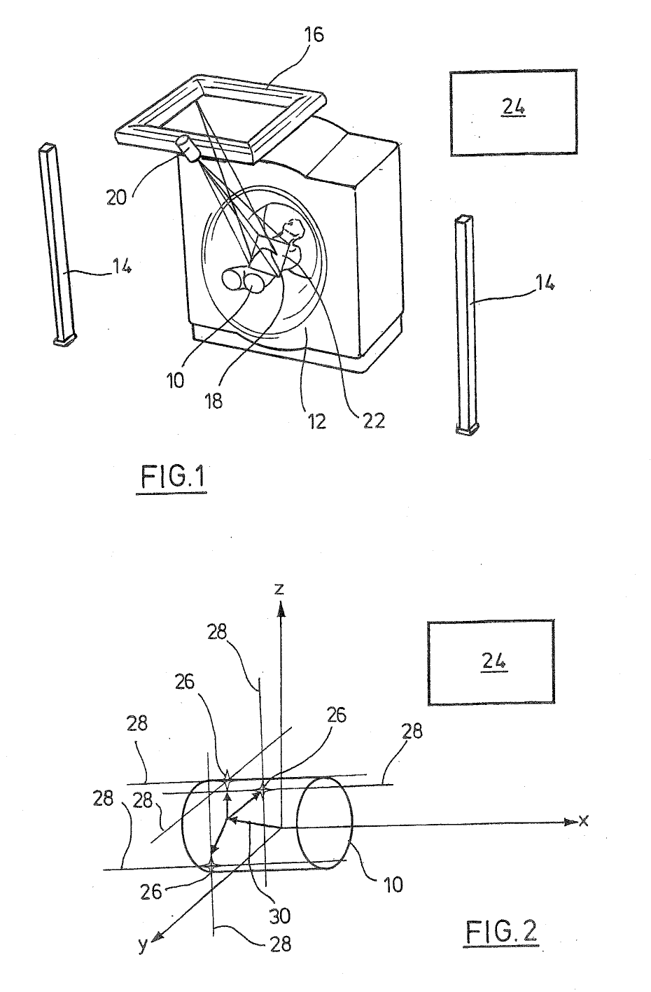

While this invention may be embodied in many different forms, there are described in detail herein a specific preferred embodiment of the invention. This description is an exemplification of the principles of the invention and is not intended to limit the invention to the particular embodiment illustrated As far as not indicated otherwise, same reference signs designate same objects in the figures. In FIG. 1, an apparatus for marking an irradiation field on the surface of a patient's body 10 represented only schematically is shown. In the realisation example in FIG. 1. the apparatus is arranged in the same room as a tomography apparatus 12 (a CT apparatus for instance) by which a virtual 3D-model of the patient's body 10 is produced for the irradiation planning. The apparatus in FIG. 1 comprises a laser system with five lasers altogether, which can be moved by motor. In this, two lasers 14 adjustable in height are provided laterally at the right and at the left side of a positioning table for the patient not shown in more detail, which each one at a time can project a horizontal line along the tomography table. In a holding device 16 situated above the tomography table, three other lasers are provided (not shown in more detail). In this, one laser is arranged to be shiftable crosswise to the table's longitudinal direction, and it projects a not shown line in the longitudinal direction of the tomography table. Two other lasers in the holder 16 are coupled with each other and project a laser line 18 to the patient's body 10, crosswise to the longitudinal direction of the tomography table. The laser system comprises further a detector device 20 in the form of a camera 20, in this case a CCD-camera 20. The field of view detected by the camera 20 is designated with 22 in FIG. 1.

By means of an analysing and control unit 24, the laser line 18 is successively guided across the surface region of interest of the patient's body 10. In doing so, the laser line 18 remains in the detection range 22 of the camera 20. The laser radiation reflected by the patient's body 10 is detected by the camera 20. From the detection result, the analysing and control unit 24 determines a 3D-model of the patient's body in the local coordinate system of the apparatus by the triangulation method, in a manner per se known. Thus, a practically infinite number of reference points on the surface of the patient's body 10 are determined in the local coordinate system assigned to the apparatus. Subsequently, the local 3D-model of the patient's body determined in such a way is compared by the analysing and control unit 24 with a virtual 3D-model of the patient's body 10, which had been produced in the context of an irradiation planning by means of mappings produced by the tomography apparatus. In particular, the distance between the two 3D-models is pointwise measured for a plurality of surface points on the envelope of the 3D-models, and the 3D-models are made to overlap by means of a suitable software such that there is a minimal distance between the 3D-models across the whole surface of the 3D-models. In this, the virtual 3D-model is suitably shifted or rotated, respectively. From the translation or rotation, respectively, of the virtual 3D-model which is necessary for this, a transformation matrix for the transformation of arbitrary coordinates from the virtual coordinate system of the irradiation planning into the local coordinate system is determined by the analysing and control unit 24. The coordinates of the irradiation isocentre and of the outline of the irradiation field determined in the context of the irradiation planing or the determined irradiation fields, respectively, are subsequently transformed into the local coordinate system by means of the transformation matrix. The transformed coordinates are then provided to the laser system. By means of a remote control for instance, the transformed coordinates can be approached by the lasers of the laser system and they can be marked by hand with a pencil on the skin of the patient for the later irradiation.

FIG. 2 shows an alternative embodiment of the apparatus of the present invention. In this case, the laser system hast at least two lasers not shown in more detail, each one of which produces one laser line at a time. Three reference points 26 have been marked on the patient's body 10 in the context of the irradiation planning. In the present case, the same are made visible, visually by means of a pencil as well as by corresponding heavy metal marks for a CT mapping of an irradiation planning. The reference points 26 can be approached by the laser system through the laser lines 28. In this, the coordinates of the reference points in the local coordinate system of the apparatus are determined by a determination of the translation of the laser lines from the zero point of the local coordinate system to the reference points 26. Thus, the coordinates of the reference points 26 in the local coordinate system are known.

In addition, the virtual coordinates of the reference points 26 in the virtual coordinate system of the 3D-model of the patient's body, which was produced in the context of the irradiation planning are also known, due to the recognizability of the CT mapping. Based on this, the transformation matrix for the transformation of arbitrary coordinates from the virtual coordinate system X, Y, Z of the virtual 3D-model of the patient's body into the local coordinate system assigned to the apparatus can be determined by means of the analysing and control unit. A corresponding displacement vector 30, representing the translation between the virtual and the local coordinate systems, is used for the transformation. The rotation parameters representing a three-dimensional rotation of the patient are determined by means of the coordinates of the reference points.

The coordinates of the isocentre and those of the outline of the calculated desired irradiation field can then be transformed into the local coordinate system assigned to the apparatus with the transformation matrix, and subsequently be provided to the laser system. In turn, the transformed coordinates can then be successively approached on the skin of the patient, by intersection of two laser lines 28 for instance, and can be marked manually with a pencil for instance.

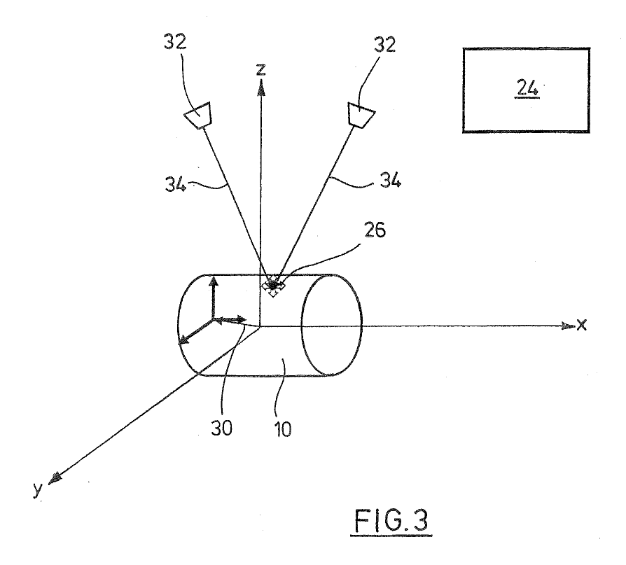

In FIG. 3, a further embodiment of the apparatus of the present invention is shown. The laser system represented in FIG. 3 has two laser projection devices 32. By an intersection of two laser beams 34, each one generated by the laser projection devices 32 at a time on the surface of the patient's body 10, reference points 26 marked on the surface of the patient's body can be approached. Due to the defined positions of the laser projection devices 32 in the room of the apparatus, the coordinates of the reference points 26 in the three-dimensional space can be determined in this way. This task is taken over by the analysing and control unit 24. As the coordinates of the reference points 26 in the virtual 3D-model of the patient's body are in turn also known from the CT irradiation planning, based on this the analysing and control unit 24 can determine the transformation matrix for the transformation of arbitrary coordinates from the virtual coordinate system into the local coordinate system. Subsequently, the coordinates of the isocentre and of the outline of the irradiation field or of the irradiation fields, respectively, can be transformed into the local coordinate system by the analysing and control unit 24 with the transformation matrix and be provided to the laser system.

In the realisation example represented in FIG. 3, the laser projection devices 32 are driven for the marking by the analysing and control unit 24, such that the irradiation field is projected to the three-dimensional surface of the patient's body 10. In this, at least one of the projection devices 32 is driven such that the laser beam 34 generated by this device 32 is guided along the transformed coordinates on the surface of the patient's body 10 sufficiently rapidly, so that the impression of a closed contour around the irradiation field results on the surface 10. The irradiation field projected on the surface of the patient's body 10 in this way can then be marked manually, for instance. When the laser system of FIG. 3 is already situated in the irradiation room used for the irradiation, the projected irradiation field can be compared even directly with the light field which visualises the beam of the irradiation apparatus. Marking the irradiation field on the surface of the patient's body by means of a pencil or the like is no more necessary in this case.

With all the apparatuses of the present invention, marking of an irradiation field representing a desired intersection field of a treatment beam with the surface of the patient's body for an irradiation therapy, for cancer treatment e.g., is possible in a simple, cost-saving and medically unobjectionable way. In this, the coordinates of the irradiation field calculated in the context of the irradiation planning by means of a virtual 3D-model of the patient, which was produced with a tomography apparatus, are transformed from the virtual coordinate system into the local coordinate system, by means of the determination of the coordinates of the reference points on the surface of the patient's body. The position change of the patient with respect to his/her position in the tomography mapping is taken into account by means of the determination of the coordinates of the reference points on the surface of the patient's body. Thus, it is not necessary to position the patient anew in the marking room. Instead, the coordinates of the irradiation field are transformed corresponding to his/her position change.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

What is claimed is:

1. An apparatus for marking an irradiation field on the surface of a patient's body, which was produced by means of a virtual 3D model of the patient's body, with a laser system for determining the coordinates of at least two reference points (26) on the surface of the patient's body (10) in a local coordinate system assigned to the apparatus, with an analysing and control unit (24) which is realised to determine a transformation matrix for the transformation of arbitrary coordinates from the virtual coordinate system into the local coordinate system from the coordinates of the reference points (26) in the local coordinate system and from coordinates of the reference points (26) in a virtual coordinate system of the virtual 3D model of the patient's body (10),
and wherein the analysing and control unit (24) is further realised to transform coordinates from the virtual coordinate system into the local coordinate system with the transformation matrix, and to provide the transformed coordinates to the laser system.

2. An apparatus according to claim 1, characterised in that the analysing and control unit (24) is realised to transform the coordinates of at least the irradiation isocentre from the virtual coordinate system into the local coordinate system, and to provide the transformed coordinates to the laser system.

3. An apparatus according to claim 1, characterised in that the analysing and control unit (24) is realised to transform the coordinates of at least the outline of the irradiation field from the virtual coordinate system into the local coordinate system with the transformation matrix, and to provide the transformed coordinates to the laser system.

4. An apparatus according to claim 1, characterised in that the analysing and control unit (24) is realised to drive the laser system such that the transformed coordinate points are approached one after the other with at least one laser beam.

5. An apparatus according to claim 1, characterised in that the laser system comprises at least two lasers, each one thereof generating one laser line (28) at a time.

6. An apparatus according to claim 1, characterised in that the local coordinates of the reference points (26) can be determined by the laser system through a determination of the translation of the laser lines (28) from the zero point of the local coordinate system to the reference points (26).

7. An apparatus according to claim 1, characterised in that the laser system comprises at least two laser projection devices (32), wherein the reference points (26) can be determined by an intersection of two laser beams (24) at a time, generated by the laser projection devices (32), on the surface of the patient's body.

8. An apparatus according to claim 7, characterised in that the analysing and control unit (24) is realised to drive the laser projection devices (32) such that the transformed coordinates can be projected to the three-dimensional surface of the patient's body (10), while at least one laser beam (34) generated by at least one of the laser projection devices (32) can be guided along the transformed coordinate points on the surface of the patient's body (10) sufficiently rapidly, so that the impression of a closed contour results on the surface.

9. An apparatus according to claim 1, characterised in that the reference points (26) are marked on the skin of the patient's body (10).

10. An apparatus according to claim 1, characterised in that a surface mapping system is provided, by which a local 3D-model of the patient's body (10) can be produced in the local coordinate system.

11. An apparatus according to claim 1, characterised in that the laser system has at least one laser generating a laser line and at least one detector device (20), wherein the analysing and control unit (24) is realised to guide a laser line (18) successively across the surface of the patient's body (10), to detect the laser light reflected by the patient's body (10) with the detector device (20) and to produce a local 3D-model of the patient's body (10) in the local coordinate system from the detected laser light.

12. An apparatus according to claim 7, characterised in that the analysing and control unit (24) is realised to determine the transformation matrix by a comparison of the local 3D-model of the patient's body (10) with the virtual 3D-model of the patient's body (10).

13. An apparatus according to claim 1, characterised in that the laser system comprises at least five lasers (14), each one thereof generating one laser line at a time, wherein two lasers (14) adjustable in the height are provided on the sides of a positioning table for the patient, which each one at a time project one horizontal line along the positioning table, and three lasers are provided above the positioning table, wherein one laser is arranged to be movable transversely to the longitudinal direction of the positioning table and projects a laser line in the longitudinal direction of the positioning table, and wherein the two other lasers are arranged to be movable in the longitudinal direction of the positioning table and project a common laser line (18) transversely to the longitudinal direction of the positioning table, being coupled with each other.

14. An apparatus according to claim 1, characterised in that the apparatus is disposed in another room than a tomography apparatus (12) used for determining the virtual 3D-model of the patient's body (10).

15. A method for marking an irradiation field on the surface of the patient's body, which was produced by means of a virtual 3D model of a patient's body, wherein the coordinates of at least two reference points (26) on the surface of the patient's body (10) are determined in a local coordinate system assigned to the apparatus, wherein a transformation matrix for the transformation of arbitrary coordinates from the virtual coordinate system into the local coordinate system is determined from the coordinates of the reference points (26) in the local coordinate system and from coordinates of the reference points (26) in a virtual coordinate system of the virtual 3D model of the patient's body (10), and wherein coordinates are transformed from the virtual coordinate system into the local coordinate system with the transformation matrix, and are provided for the marking.

16. A method according to claim 15, characterised in that the coordinates of at least the irradiation isocentre are transformed from the virtual coordinate system into the local coordinate system with the transformation matrix, and are provided for the marking.

17. A method according to claim 15, characterised in that the coordinates of at least the outline of the irradiation field are transformed from the virtual coordinate system into the local coordinate system with the transformation matrix, and are provided for the marking.

18. A method according to claim 15, characterised in that the transformed coordinate points are approached one after the other with at least one laser beam.

19. A method according to claim 15, characterised in that the laser system comprises at least two lasers, each one thereof generating one laser line (28) at a time.

20. A method according to claim 19, characterised in that the local coordinates of the reference points (26) are determined by a determination of the translation of the laser lines (28) from the zero point of the local coordinate system to the reference points (26)

21. A method according to claim 15, characterised in that the reference points (26) are determined by an intersection of two laser beams (24) at a time, generated by at least two laser projection devices (32), on the patient's surface (10).

22. A method according to claim 21, characterised in that the transformed coordinates are projected to the three-dimensional surface of the patient's body (10) while at least one laser beam (34) generated by at least one of the laser projection devices (32) is guided along the transformed coordinate points on the surface of the patient's body (10) sufficiently rapidly, so that the impression of a closed contour results on the surface.

23. A method according to claim 15, characterised in that the reference points (26) are marked on the skin of the patient's body (10) in the production of the virtual 3D-model of the patient's body (10).

24. A method according to claim 15, characterised in that a local 3D-model of the patient's body (10) is produced in the local coordinate system by a surface mapping system.

25. A method according to claim 15, characterised in that a laser line (18) is guided successively across the surface of the patient's body (10) with at least one laser, the laser light reflected by the patient's body (10) is detected by at least one detector device (20) and a local 3D-model of the patient's body (10) in the local coordinate system is produced from the detected laser light.

26. A method according to claim 25, characterised in that the transformation matrix is determined by a comparison of the local 3D-model of the patient's body (10) with the virtual 3D-model of the patient's body (10).

27. A method according to claim 15, characterised in that at least five lasers (14) are provided, each one generating one laser line at a time, wherein two lasers (14) adjustable in the height are provided on the sides of a positioning table for the patient, which each one at a time project one horizontal line along the positioning table, and the remaining lasers are provided above the positioning table, wherein one laser is arranged to be movable transversely to the longitudinal direction of the positioning table and projects a laser line in the longitudinal direction of the positioning table, and wherein the two other lasers are arranged to be movable in the longitudinal direction of the positioning table and project a common laser line (18) transversely to the longitudinal direction of the positioning table, being coupled with each other.

28. A method according to claim 15, characterised in that the method is performed in another room than the determination of the virtual 3D-model of the patient's body (10).
